(19) United States
(12) Patent Application Publication
(10) Pub. No.: US 2009/0042317 A1
(43) Pub. Date: Feb. 12, 2009

(54) SUBSTANCE IMMOBILIZING APPARATUS, SUBSTANCE DETECTING APPARATUS AND SUBSTANCE IMMOBILIZING METHOD

(75) Inventor: Takashi Ikeda, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/180,391

(22) Filed: Jul. 25, 2008

(30) Foreign Application Priority Data
Aug. 9, 2007 (JP) 2007-208150

(51) Int. Cl. G01N 33/543; B01J 19/00; G01N 21/64

(52) U.S. Cl. 436/518; 422/68.1; 422/82.01; 422/82.08

(57) ABSTRACT

The efficiency of the specific binding of a target substance to an immobilization region is increased. As a first step, target substance 1001 is drawn to immobilization region 1003 on which immobilized substance 1002 to which target substance 1001 can specifically bind is immobilized. As a second step, only target substance 1001 that does not specifically bind is drawn away from immobilization region 1003. As a third step, target substance 1001 is drawn to immobilization region 1003 again. By alternately repeating the second step and the third step to contact target substance 1001 with immobilized substance 1002 a plurality of times, target substance 1001 is specifically bound to immobilized substance 1002.

### BACKGROUND OF THE INVENTION

1. Field of the Invention

The present invention relates to a substance immobilizing apparatus for immobilizing a substance in a sample to a solid phase, and a substance detecting apparatus having the apparatus. Further, the present invention relates to a substance immobilizing method for immobilizing a substance in a sample to a solid phase.

2. Description of the Related Art

Technique of immobilizing a substance present in a liquid to the desired region is important in genome analysis and immunoassay. For example, in immunoassay, the binding between binding substance (substance that specifically binds to the target substance) immobilized on a substrate and the target substance requires a long time to reach an equilibrium state, which is a big factor that prevents the shortening of examination time. In a method which has been known from long ago, magnetic particles are bound to a target substance, a magnetic field is applied, and the target substance is efficiently drawn onto a substrate, on which a binding substance is immobilized. (see U.S. Pat. No. 4,847,193). The external force used for drawing the target substance need not be a magnetic field, and any external force, for example, an electric field and a centrifugal force can be used as long as the target substance can be drawn to the desired site.

The method of drawing the target substance to the immobilization region by some external force as described above largely contributes to the shortening of time during which the substance is immobilized. But, since the target substance is oriented in various directions, there are many cases where even when the target substance and the binding substance approach each other, the binding site of the target substance and the binding site of the binding substance do not contact and bind. Therefore, only by drawing the target substance to the immobilization region, the efficiency of specific binding is not sufficient.

### SUMMARY OF THE INVENTION

In view of the above prior art problem, it is an object of the present invention to provide a substance immobilizing apparatus and substance immobilizing method that increase the efficiency of the specific binding of a target substance to an immobilization region.

In order to achieve the above object, the present invention is characterized by a substance immobilizing apparatus having a region on which an immobilized substance to which a target substance can specifically bind is immobilized, including an external force applying unit for applying an external force for moving the target substance back and forth in the direction perpendicular to the in-plane direction of the region and contacting the target substance with the immobilized substance a plurality of times so as to specifically bind the target substance to the immobilized substance.

Also, the present invention is characterized by including: the stage of preparing a container having a region on which an immobilized substance to which a target substance can specifically bind is immobilized, and a sample including the target substance; the first step of injecting the sample into the container and drawing the target substance to a surface of the region; the second step of moving the target substance that does not specifically bind to the immobilized substance when the target substance is drawn to the surface of the region in the first step, in the direction of drawing the target substance that does not specifically bind to the immobilized substance away from the surface of the region; and the third step of specifically binding the target substance to the immobilized substance by drawing the target substance to the surface side of the region again to contact the immobilized substance.

Further features of the present invention will become apparent from the following description of exemplary embodiments with reference to the attached drawings.

### BRIEF DESCRIPTION OF THE DRAWINGS

FIG. 1 is a conceptual view describing the main steps of the substance immobilizing method of the present invention.

FIGS. 2A and 2B are conceptual views describing a method of promoting antigen-antibody reaction using a charge. FIG. 2A is a conceptual view for describing the step of drawing an antigen to an immobilization region. FIG. 2B is a conceptual view for describing the step of drawing the antigen away from the immobilization region.

FIG. 3 is a conceptual view for describing a magnetic field generated from a coil.

FIG. 4 is a conceptual view describing a substance immobilizing apparatus that is one embodiment of the present invention.

FIGS. 5A, 5B, 5C and 5D are conceptual views describing one embodiment of the substance immobilizing method of the present invention. FIG. 5A is a conceptual view describing the step of making a complex of a target substance and a carrier. FIG. 5B is a conceptual view for describing a state in which the complex is drawn to an immobilization region. FIG. 5C is a conceptual view describing the step of drawing the complex away from the immobilization region. FIG. 5D is a conceptual view describing the step of drawing the complex to the immobilization region again.

FIG. 6 is a conceptual view illustrating a cross section of magnetoresistance effect devices used in one embodiment of the present invention.

FIG. 7 is a conceptual view describing the state of a bias magnetic field applied to a magnetic bead and a magnetic stray field generated from the magnetic bead.

FIG. 8 is a conceptual view describing a substance detecting apparatus including a substance immobilizing apparatus that is one embodiment of the present invention.

FIG. 9 is a conceptual view describing a substance detecting apparatus including a substance immobilizing apparatus that is one embodiment of the present invention.

FIG. 10 is a conceptual view describing the state of a complex in which DNAs bind.

### DESCRIPTION OF THE EMBODIMENTS

Embodiments of the present invention will be described below with reference to the drawings.

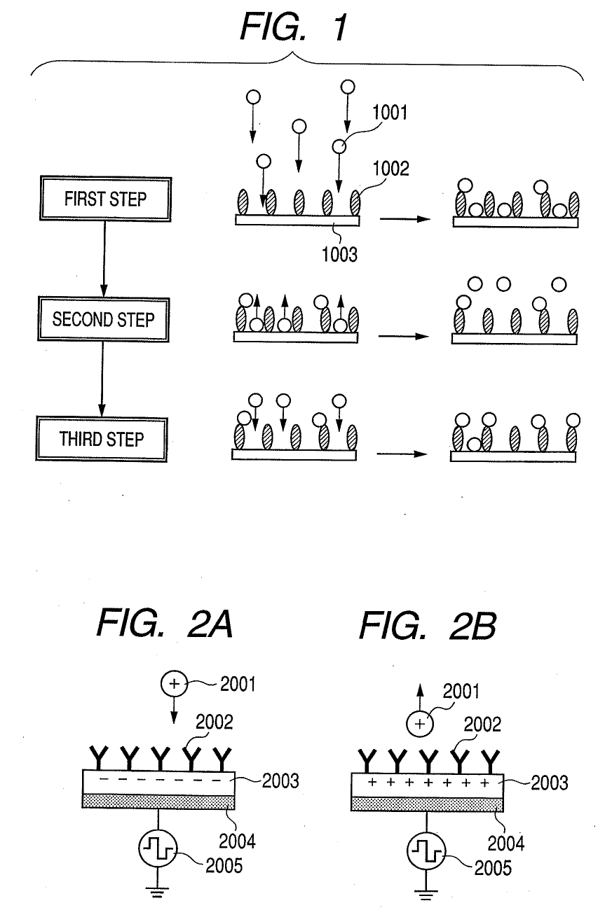

FIG. 1 is a view illustrating main steps in the substance immobilizing method of the present invention. The main steps in the substance immobilizing method of the present invention include the following three steps. As a first step, by applying an external force of sufficient magnitude to the entire sample including target substance 1001, target substance 1001 is drawn to immobilization region 1003. Immobilized substance 1002 capable of specifically binding to target substance 1001 is previously immobilized on immobilization region 1003. Therefore, when target substance 1001 is drawn as described above, part of target substance 1001 is specifically bound to immobilized substance 1002. As a second step, among target substance 1001 that is drawn to immobilization region 1003, target substance 1001 that does not specifically bind to immobilized substance 1002 is drawn away from immobilization region 1003. The external force applied at this time should be of such magnitude that target substance 1001 specifically bound to immobilized substance 1002 is not drawn away. As a third step, an external force similar to the external force in the first step is applied to draw target substance 1001 to immobilization region 1003 again.

By alternately carrying out the above second step and third step a plurality of times, target substance 1001 may be moved back and forth in the direction perpendicular to the in-plane direction of immobilization region 1003 (hereinafter, abbreviated as the perpendicular-to-plane direction). By such back-and-forth movement, target substance 1001 is contacted with immobilized substance 1002 a plurality of times, so that the efficiency of specific binding, that is, binding reaction can be more improved.

The present invention can be applied to target substance 1001 as long as target substance 1001 is specifically bound to immobilized substance 1002 on immobilization region 1003. For example, an antibody is immobilized on the surface of immobilization region 1003 as immobilized substance 1002, and target substance 1001 can be an antigen that specifically binds to the antibody. Alternatively, DNA is immobilized on the surface of immobilization region 1003 as immobilized substance 1002, and complementary DNA that specifically binds to the DNA may be used as target substance 1001.

Immobilization region 1003 may be the side surface or bottom surface of a container in which the sample is kept, and may be one place or a plurality of places. For example, by providing immobilization regions 1003 on two parallel side surfaces in a container and moving target substance 1001 back and forth between immobilization regions 1003, target substance 1001 can be bound to immobilized substance 1002 that is immobilized on the surface of both immobilization regions 1003. In this case, specific binding for a shorter time than the case where immobilization region 1003 is one place is possible.

By immobilization region 1003 being the surface of a sensor used for the detection of target substance 1001, target substance 1001 that is immobilized can be detected.

When impurities are present in the sample, the quality of the detection and recovery of target substance 1001 can be enhanced by removing the impurities from immobilization region 1003 after the final third step for substance immobilization is finished.

### (Regarding External Force for Moving Target Substance)

A centrifugal force, for example, can be used for the external force used in the present invention. In other words, by rotating the container in which the sample is kept, target substance 1001 present in the sample can be relatively moved with respect to immobilization region 1003. By changing the relative position of the rotation axis and the container, the direction in which the centrifugal force acts can be changed. Alternatively, when target substance 1001 is charged, target substance 1001 can also be moved by an electrostatic force. Alternatively, when the target substance is magnetized, target substance 1001 can be moved by using a magnetic field. Of course, target substance 1001 may be moved by combining a plurality of these forces.

(Regarding Another External Force for Moving Target substance in In-Plane Direction of Immobilization Region)

In addition to the back and forth movement in the perpendicular-to-plane direction of the plane of immobilization region 1003, target substance 1001 may be gradually moved on the surface of immobilization region 1003 in the direction parallel to the plane of immobilization region 1003. By this movement, target substance 1001 can also be specifically bound to immobilized substance 1002 on immobilization region 1003 uniformly. As such an external force, external forces, such as an electrostatic force, a magnetic force and a centrifugal force, may be used. Also, a hydraulic pressure may be applied such that a liquid including target substance 1001 moves on the surface of immobilization region 1003.

(Example of Substance Immobilizing Method of the Present Invention)

FIGS. 2A and 2B are views illustrating an embodiment of the substance immobilizing method of the present invention when positively charged antigen 2001 is used as a target substance, and antibody 2002 is used as an immobilized substance. Antibody 2002 is immobilized on immobilization region 2003 on electrode 2004. Electrode 2004 is connected to power supply 2005 and is adapted such that the polarity can be changed. By doing so, a positive or negative charge can be provided to immobilization region 2003.

As a first step, immobilization region 2003 is negatively charged. Then, antigen 2001 that is present dispersed in the sample is drawn to immobilization region 2003 by an electrostatic force (see FIG. 2A). At this time, antigen 2001 contacting with antibody 2002 specifically binds to antibody 2002 by antigen-antibody reaction.

As a second step, after antigen 2001 is drawn to immobilization region 2003, immobilization region 2003 is positively charged. Then, a repulsive force is generated between antigen 2001 and immobilization region 2003, and antigen 2001 that does not specifically bind to antibody 2002, and positively charged impurities move away from immobilization region 2003 (see FIG. 2B).

As a third step, by negatively charging immobilization region 2003 again, antigen 2001 is again drawn to immobilization region 2003.

By alternately carrying out the above second step and third step a plurality of times to move antigen 2001 back and forth, antigen 2001 can be contacted with antibody 2002 a plurality of times. By this substance immobilizing method, more antigen 2001 can be specifically bound to antibody 2002.

### (Use of Carrier for Movement of Target Substance)

In a case that the physical quantity used for moving the target substance is small, and the magnitude of the external force that can be applied to the target substance is insufficient, some substance having a sufficiently large physical quantity, as a carrier, may be bound to the target substance to form a complex. Any carrier can be used as long as an external force can be applied and the complex can be moved. Examples of the carrier include charged substances, magnetic materials, and substances having a large mass. Further, in order to bind the target substance to the carrier, the surface of the carrier may be covered with a substance that easily binds to the target substance.

As an example of the above, the carrier is a magnetic bead having a surface covered with an antibody. An antigen that is a target substance is bound to the carrier by antigen-antibody reaction. Subsequently, when a magnetic force having such a magnetic field distribution that the strength increases as approaching the immobilization region is applied to the carrier, the antigen is drawn to the immobilization region. Then, when the applied magnetic field is changed to have such a magnetic field distribution that the strength increases in the direction away from the immobilization region, the antigen moves away from the immobilization region. By moving the complex including the antigen and the carrier back and forth and repeatedly contacting the complex with an antibody immobilized on the immobilization region by alternately applying magnetic fields having different magnetic field strength distributions in this way, the probability of specific binding by antigen-antibody reaction can be increased.

(Consideration of Magnitude of External Force When Drawing Target Substance Away from Immobilization Region)

In the second step, in order to draw the target substance that is not specifically bound away from the immobilization region so that the target substance that is specifically bound to the immobilized substance does not dissociate, it is necessary that the external force required to move the target substance is sufficiently smaller than the binding force of specific binding. This respect is described using, as an example, the case where an antigen as a target substance, an antibody as an immobilized substance, and a magnetic bead as a carrier are used.

Now, performing the substance immobilizing method of the present invention in a sample liquid injected into a container is considered. The immobilization region is in the bottom portion of the container. When the mass and volume of a complex including a magnetic bead, an antibody and an antigen are m and V respectively, and the density of the sample liquid is ρB, gravity F_{G} and buoyancy F_{B} acting on the still standing complex are respectively as shown by the following formulas (1) and (2):

F_{G}=gm (1)

F_{B}=gρ_{B}V (2)

Here, g is the acceleration of gravity. In order to draw the complex away from the immobilization region, that is, draw the complex upward from the immobilization region, it is necessary to apply a force larger than the force obtained by subtracting buoyancy F_{B} from gravity F_{G}. For example, when the mass of the complex is 6.0×10⁻¹⁵ [kg], the volume of the complex is 12.6 [μm³], and the density of the sample liquid is 1002.8 [kg/m³], gravity F_{G} is about 6×10⁻¹⁴ [N], and buoyancy F_{B} is about 4×10⁻¹⁴ [N]. Therefore, the force necessary to draw this complex up should be a force of more than 2×10⁻¹⁴ [N].

An example of moving the above complex by a magnetic force is considered. Force F_{H} acting on the magnetic bead, in which the magnitude of magnetization is M, in magnetic field H is expressed by formula (3) shown below:

F_{H}=grad(M·H). (3)

Here, if the magnetic bead is magnetized even when a magnetic field is not applied, the complex aggregates by magnetostatic binding in the sample liquid. The aggregate complex becomes difficult to specifically bind to the antibody immobilized on the immobilization region, and causes problems, such as a decrease in the efficiency of the specific binding by antigen-antibody reaction, therefore, the aggregate complex is not favorable. Then, as the magnetic bead used in the present invention, magnetic beads including a material exhibiting superparamagnetism are favorable. In other words, only when a magnetic field is applied to the complex to move the antigen, the magnetic bead is magnetized, and when the application of the magnetic field is stopped, the magnetic bead behaves as a nonmagnetic material. Magnetization M in superparamagnetism is expressed by a function of magnetic field H, and in a small magnetic field region, the relationship between magnetization and the magnetic field is substantially linear. In other words, the magnetization M of the magnetic bead that is superparamagnetic is expressed by M=χH (χ is susceptibility) in a small magnetic field region. As the magnetic field is increased, the magnetization of the substance exhibiting superparamagnetism is finally saturated.

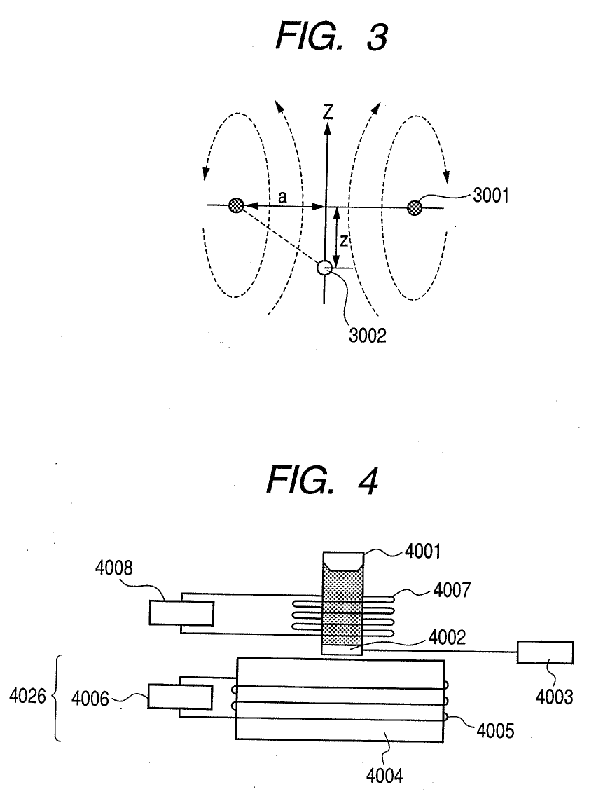

The distribution of magnetic field strength for magnetic field H depends on the magnetic field applying unit. FIG. 3 illustrates a magnetic field applying unit used in this example. In this example, coil 3001 having radius a is used, and magnetic bead 3002 is present in a position apart by distance z on the center line of this coil 3001. By flowing current through this coil 3001 and generating a magnetic field, an external force is provided to the magnetic bead. When current I is flowed through coil 3001, magnetic field H generated on the center line of the coil is expressed by the following formula (4):

$$H = \frac{{Ia}^{2}}{2 {( {a}^{2} + {z}^{2} )}^{\frac{3}{2}}} \qquad ( 4 )$$

In other words, when small magnetic field H is applied to magnetic bead 3002 exhibiting superparamagnetism by the above coil 3001, magnetic force F_{H} acting on magnetic bead 3002 is expressed by the following formula (5):

$${F}_{H} = \frac{{3}_{X} {I}^{2} {a}^{4} z}{2 {( {a}^{2} + {z}^{2} )}^{4}} \qquad ( 5 )$$

For example, when the radius a of coil 3001 is 1 [mm], distance z from the center of coil 3001 to magnetic bead 3002 is 0.5 [mm], and current I is 1.7 [A] or more, a magnetic force F_{H} of about 2×10⁻¹⁴ [N] is applied to magnetic bead 3002. Therefore, in the above example, magnetic bead 3002 can be drawn up against the weight.

In the above example, in order that the specific binding of the target substance is maintained when the target substance is drawn away from the immobilization region, the binding force of the specific binding should be a force sufficiently larger than 2×10⁻¹⁴ [N]. For example, the binding force of the specific binding of biotin and an anti-biotin antibody is 1.5×10⁻¹³ [N] or more, and the present invention can be sufficiently used for this binding force of the specific binding according to the above example.

Further, in order to carry out the above example with a smaller current, the winding number of coil 3001 may be increased. By increasing the winding number of coil 3001, a large magnetic field can be generated even with a small current, so that magnetic bead 3002 can be moved. In this way, when coil 3001 is used for a magnetic field applying unit, the magnitude of the magnetic force can be easily controlled by current. Also, the magnetic force may be controlled by using a permanent magnet as a unit for generating magnetic field H, and controlling the distance between the magnetic bead and the permanent magnet.

(Description of Detecting Method)

Various physical quantities, such as light, static electricity, a magnetic field and radiation, can be used for a method for detecting a target substance that is specifically bound. When the physical quantity of the target substance cannot be directly detected, the target substance can be indirectly detected by binding a detectable labeled substance having a large physical quantity to the target substance and detecting the physical quantity of the labeled substance. Therefore, both a carrier and a labeled substance may be bound to the target substance. When a magnetic bead is used as a carrier as in the above-described example, the magnetic bead can also be used as a labeled substance for detection. In this case, a magnetic stray field generated from the magnetic bead is detected by a magnetic sensor. Various sensors, such as superconducting quantum interference devices (SQUID), magnetoresistance effect devices, magnetic impedance devices, flux gates, Hall devices and coils, can be used for the magnetic sensor.

Also for a method for detecting a target substance by light, various methods can be used. By irradiating a target substance and a labeled substance bound to the target substance with light from outside and measuring the transmitted light and reflected light or the intensity of plasmon, the detection of the target substance can be performed. Alternatively, when one of the target substance and the labeled substance includes a phosphor, the detection of the target substance is also possible by measuring light emitted from the phosphor. Alternatively, when one of the target substance and the labeled substance includes a light emitting substrate, the detection of the target substance is also possible by measuring light emitted from the light emitting substrate.

Also, when one of the target substance and the labeled substance includes a radionuclide, the detection of the target substance is possible using a radiation detecting device.

Also, when one of the target substance and the labeled substance is charged, a method for detecting the target substance using a field effect transistor (FET) is mentioned. The gate electrode portion of the FET is used as an immobilization region, and an immobilized substance that specifically binds to a substance to be measured is immobilized on the surface of the immobilization region. Then, one of the target substance that is charged and a complex of the target substance and a labeled substance is bound to the immobilized substance by the substance immobilizing method of the present invention. Then, the current flowing between the source and drain of the field effect transistor is changed by an electric field generated by the charge. By measuring the change in current, the detection of the target substance is possible.

(Specific Example of Apparatus)

Next, apparatuses for carrying out the above-described substance immobilizing method and substance detecting method are illustrated.

### Embodiment 1

FIG. 4 is a configuration diagram illustrating Embodiment 1 of a substance detecting apparatus including a substance immobilizing apparatus of the present invention.

The substance immobilizing apparatus of Embodiment 1 includes cylinder-shaped container 4001 and an electromagnet 4026 located under container 4001. This electromagnet 4026 that is a first external force applying unit includes iron core 4004 having a circular cross section with the diameter five times larger than the diameter of the bottom surface of container 4001, coil 4005 wound around iron core 4004, and power supply 4006 that can flow a variable current through coil 4005. Also, a large number of magnetoresistance effect devices 4002 that are magnetic sensors are provided on the bottom surface of container 4001 as immobilization regions for a target substance. Magnetoresistance effect devices 4002 are connected to external detection circuit 4003. Further, circular coil 4007 parallel to the bottom surface of container 4001 is fixed above the bottom surface of container 4001. This circular coil 4007 is a second external force applying unit and is connected to power supply 4008 for generating a magnetic field. Also, the substance immobilizing apparatus is adapted such that the distance between the electromagnet 4026 located under container 4001 and container 4001 can be changed so that magnetic fields having various magnitudes can be applied.

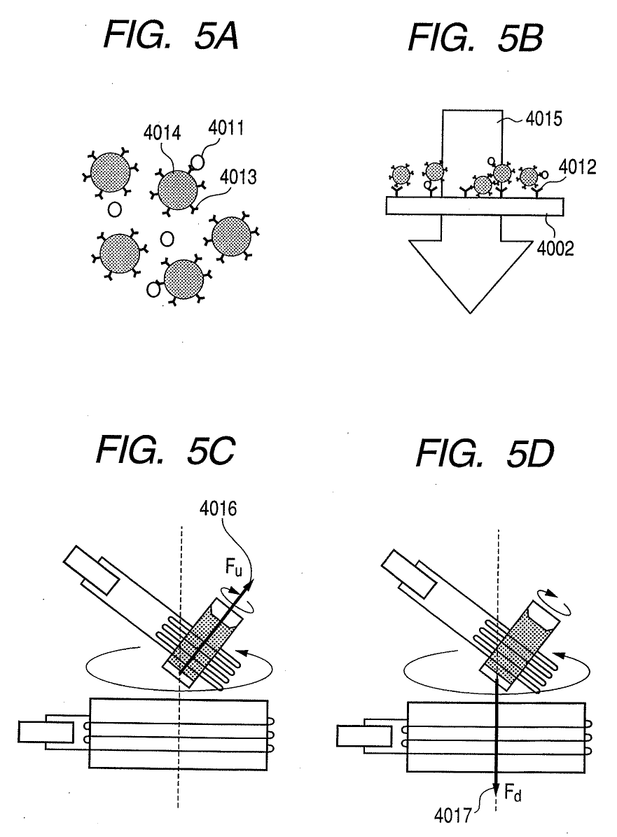

Next, a substance immobilizing method using the substance immobilizing apparatus as described above is described. Prostate specific antigen (PSA) 4011 that is a biological substance is used as a target substance, and primary antibody 4012 to which PSA 4011 can specifically bind is immobilized on the surface of magnetoresistance effect devices 4002. Also, magnetic beads 4014 covered with secondary antibody 4013 that is a substance capable of specifically binding to PSA 4011 are used as a carrier also serving as a labeled substance (see FIG. 5A).

First, before the first step of the present invention is performed, magnetic beads 4014 covered with secondary antibody 4013, and PSA 4011 are bound in a liquid to form complexes. This reaction may be performed in the above container 4001, or the complexes may be formed in a different container and then injected into the above container 4001.

Next, as a first step, magnetic field 4015 is applied by the electromagnet 4026 to draw magnetic beads 4014 dispersed in the liquid to the bottom surface of the container. When magnetic beads 4014 are drawn to the bottom surface of the container, among magnetic beads 4014 forming complexes with PSA 4011, magnetic beads 4014 contacting with primary antibody 4012 are specifically bound to primary antibody 4012 by antigen-antibody reaction (see FIG. 5B).

As a second step, when magnetic beads 4014 are drawn to the bottom surface of the container, the application of magnetic field 4015 by the electromagnet 4026 is stopped. Then, by tilting container 4001 with respect to the surface of iron core 4004 of the electromagnet 4026 and rotating and revolving container 4001 around the central axis of iron core 4004, an external force that moves magnetic beads 4014 in the in-plane direction of the bottom surface of the container is applied to magnetic beads 4014. At this time, by applying magnetic field Fᵤ 4016 by circular coil 4007, such an external force that draws magnetic beads 4014 away from magnetoresistance effect devices 4002 is applied. Of course, the magnetic force provided to magnetic beads 4014 at this time is of such magnitude that specific binding to primary antibody 4012 is not broken (see FIG. 5C).

As a third step, when unbound magnetic beads float from magnetoresistance effect devices 4002 after the second step, the application of magnetic field Fᵤ 4016 by circular coil 4007 is stopped, and magnetic field F_{d} 4017 is applied by the electromagnet 4026 again (see FIG. 5D). By this step, the magnetic beads are drawn rather obliquely to the surface of magnetoresistance effect devices 4002. However, at this time, magnetic field F_{d} 4017 applied by the electromagnet is smaller than magnetic field 4015 applied by the electromagnet 4026 when magnetic beads 4014 are first drawn to magnetoresistance effect devices 4002.

By alternately carrying out the above second step and third step a plurality of times, magnetic beads 4014 contact with primary antibody 4012 a plurality of times while gradually moving in the in-plane direction of magnetoresistance effect devices 4002 on the surface of magnetoresistance effect devices 4002. Therefore, PSA 4011 in the complexes and primary antibody 4012 specifically bind more efficiently, so that higher antigen-antibody reaction efficiency can be achieved.

Complexes that do not specifically bind by antigen-antibody reaction are floated from magnetoresistance effect devices 4002 and then removed together with the buffer solution from container 4001. By replacing the buffer solution with a pure buffer solution that does not include impurities several times, the complexes that do not specifically bind can be removed. Alternatively, also by collecting the complexes that do not specifically bind to primary antibody 4012 and unnecessary magnetic beads 4014 that do not form complexes with PSA 4011 by a magnetic force and removing the complexes and unnecessary magnetic beads 4014 from the container, the removal of unnecessary magnetic beads 4014 can be achieved.

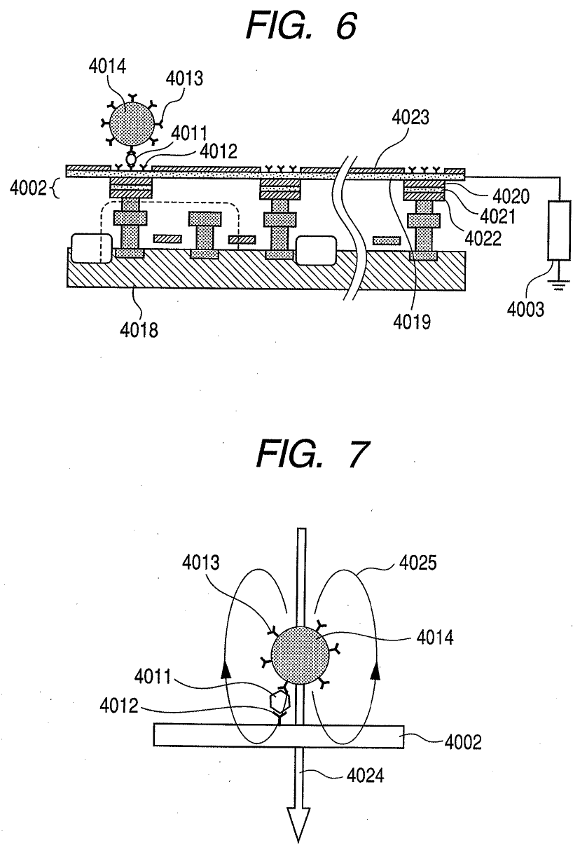

Further, the configuration of the substance detecting apparatus of this embodiment is described in detail. Here, the case where magnetic beads 4014 in the specifically bound complexes are detected using the magnetoresistance effect devices by the above substance immobilizing method is described. FIG. 6 is a cross-sectional view illustrating the structure of spin tunnel magnetoresistance effect devices 4002 used in this Embodiment 1. Magnetoresistance effect devices 4002 on the bottom surface of container 4001 is magnetoresistance effect devices 4002 having an area of 1 μm×2 μm. The films of magnetoresistance effect device 4002 include Ta/CuN/Ta/MnPt/CoFe/Ru/CoFeB/MgO/CoFeB/Ru/Au. Magnetoresistance effect device 4002 roughly includes, in order from the upper layer, detection layer 4020, tunnel film 4021 and magnetized pinned layer 4022. One select transistor 4018 is connected to one magnetoresistance effect device 4002, and a plurality of magnetoresistance effect devices 4002 are commonly connected to upper electrode 4019. Also, the upper electrode is covered with SiN insulating film 4023 except directly on the surface of magnetoresistance effect devices 4002. Primary antibody 4012 is immobilized only directly on the surface of the magnetoresistance effect devices that is not covered with SiN insulating film 4023. Therefore, the complexes are immobilized only on the surface of magnetoresistance effect devices 4002.

Next, a substance detecting method using such a substance detecting apparatus is described. In this Embodiment 1, specifically binding complexes are indirectly detected by detecting magnetic beads 4014. As magnetic beads 4014 used here, magnetic beads having a diameter of 200 nm and exhibiting superparamagnetism are used. Therefore, magnetic beads 4014 do not generate a magnetic field in a nonmagnetic field. In order to detect such magnetic beads 4014 by magnetoresistance effect devices 4002, magnetic beads 4014 need to be magnetized. A general method for the magnetization includes the application of a bias magnetic field. The change in the resistance value of magnetoresistance effect devices 4002 is insensitive to a magnetic field perpendicular to the film plane of the magnetoresistance effect devices and sensitive to a magnetic field in the in-plane direction of the film. Then, by applying bias magnetic field 4024 perpendicular to the film plane so that the resistance value of magnetoresistance effect devices 4002 are not changed, magnetic beads 4014 are magnetized. Here, the magnitude of bias magnetic field 4024 can be such magnitude that the magnetization of the magnetic beads used is not saturated. Such magnitude of the magnetic field is about 500 [Oe] to 2000 [Oe]. Magnetic stray field 4025 having a component in the in-plane direction of the film of the magnetoresistance effect device 4002 is generated by magnetic bead 4014 that is magnetized by the bias magnetic field. The resistance value of magnetoresistance effect device 4002 is changed by this magnetic stray field 4025 (see FIG. 7). When detecting magnetic beads 4014, select transistors 4018 are sequentially turned ON to detect the resistance value of each magnetoresistance effect device, and based on these detected values, the quantity of binding PSA 4011 is indirectly detected.

### Embodiment 2

In this embodiment, an example using a centrifugal force as a way of moving a target substance in the in-plane direction of an immobilization region is described. FIG. 8 is a configuration diagram illustrating Embodiment 2 of a substance detecting apparatus including a substance immobilizing apparatus of the present invention. The substance immobilizing apparatus of this Embodiment 2 includes container 8001, circular coil 8009 located under container 8001, and an electromagnet 8011 located under container 8001. Magnetoresistance effect devices 8002 similar to the magnetoresistance effect devices of Embodiment 1 are provided on the bottom surface of container 8001 as immobilization regions for a target substance.

These magnetoresistance effect devices 8002 are connected to external detection circuit 8003. Further, above the bottom surface of container 8001, circular coil 8007 parallel to the bottom surface is installed. This circular coil 8007 is connected to power supply 8008. Container 8001 includes a mechanism that enables container 8001 to rotate around the central axis.

The electromagnet 8011 located under container 8001 includes iron core 8004 having a pointed upper tip, and coil 8005 and 8009 wound around the iron core, and is located so that the center line is aligned with the center line of container 8001. Further, this electromagnet 8011 includes a mechanism that can move the electromagnet away from container 8001. Coil 8005 and circular coil 8009 are connected to power supplies 8006 and 8010 respectively.

Further, a substance immobilizing method using the substance immobilizing apparatus as described above is described in detail. Here, as in Embodiment 1, the case where magnetic beads covered with a secondary antibody are used as a carrier and labeled substance and where PSA is used as a target substance is described.

First, as in Embodiment 1, magnetic beads covered with a secondary antibody, and PSA are bound to form complexes.

As a first step, by flowing current through coil 8005 by power supply 8006 to allow the electromagnet to generate a magnetic force, the complexes are collected around the center of the bottom portion of container 8001. Subsequently, the electromagnet 8011 is moved away from container 8001.

As a second step, current is flowed through circular coil 8007 to draw the magnetic beads away from magnetoresistance effect devices 8002.

As a third step, current is flowed through circular coil 8009 to draw the magnetic beads to magnetoresistance effect devices 8002 again.

By alternately repeating the above second step and third step, the complexes are moved up and down near magnetoresistance effect devices 8002. Further, with these steps, container 8001 is rotated. In other words, complexes that are not immobilized on the surface of magnetoresistance effect devices 8002 in the lower portion of container 8001 move in the direction of the side surface of container 8001 from the center of container 8001 by a centrifugal force, while moving up and down.

In this process, magnetic beads to which PSA binds to form complexes are specifically bound by antigen-antibody reaction before reaching the side surface of container 8001. Magnetic beads that reach the side surface of container 8001 without being specifically bound may be removed by subsequently replacing the buffer solution. More favorably, such location that no magnetoresistance effect devices 8002 are near the side surface of container 8001 is provided so that magnetic beads reaching the side surface of container 8001 are not detected by external detection circuit 8003

### Embodiment 3

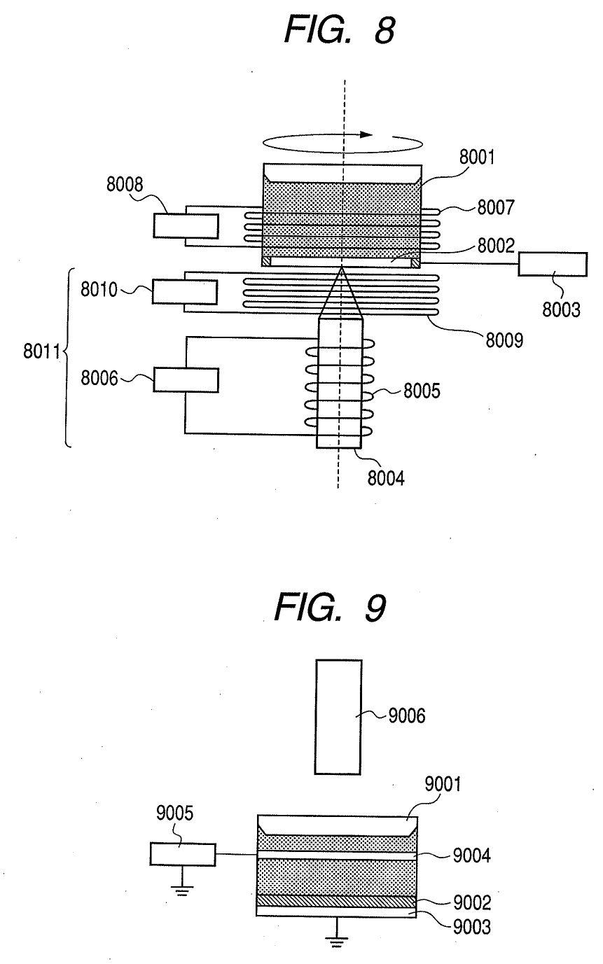

FIG. 9 is a configuration diagram illustrating Embodiment 3 of a substance detecting apparatus including a substance immobilizing apparatus of the present invention. The substance immobilizing apparatus of this Embodiment 3 includes container 9001, and lower electrode 9003 and upper electrode 9004 located in the bottom portion and upper portion of container 9001 respectively. By connecting power supply 9005 to upper electrode 9004 and grounding lower electrode 9003, an electric field can be formed between both electrodes. Also, the substance immobilizing apparatus is adapted such that the polarity of upper electrode 9004 and lower electrode 9003 can be reversed. Immobilization region 9002 is on the surface of lower electrode 9003, and an immobilized substance to which a target substance can specifically bind is immobilized on immobilization region 9002.

Also, in order to detect the target substance, photomultiplier 9006 for measuring light is installed above container 9001 in this Embodiment 3.

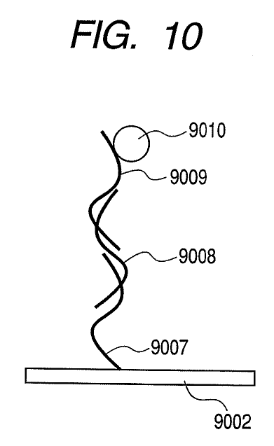

Further, such a substance immobilizing method that uses the substance immobilizing apparatus as described above and specifically binds DNA that is a biological substance is described in detail. Here, the case where target DNA 9008 is used as a target substance and where immobilized DNA 9007 that is a substance capable of specifically binding to target DNA 9008 is used as an immobilized substance is described (see FIG. 10).

As a first step, a sample including target DNA 9008 is put in the above container 9001, and by setting the voltage of power supply 9005 to a negative voltage, upper electrode 9004 is a negative electrode and lower electrode 9003 is a positive electrode. Since DNA has a negative charge, the DNA in the sample moves toward immobilization region 9002. When target DNA 9008 is present in the sample, target DNA 9008 contacting with immobilized DNA 9007 is specifically bound in this step.

As a second step, the polarity of lower electrode 9003 and upper electrode 9004 is reversed. Then, target DNA 9008 that is not specifically bound to immobilized DNA 9007 moves above immobilization region 9002. However, the voltage applied between the electrodes at this time is adjusted such that target DNA 9008 that already specifically binds does not dissociate.

As a third step, when target DNA 9008 that does not specifically bind slightly floats from immobilization region 9002, the polarity of the electrodes is reversed again to draw target DNA 9008 to immobilization region 9002 again.

By alternately carrying out the second step and third step a plurality of times to move the DNA in the sample up and down, target DNA 9008 is contacted with immobilized DNA 9007 a plurality of times. The probability that target DNA 9008 specifically binds to immobilized DNA 9007 can be increased by these steps.

Next, a method for detecting target DNA 9008 that is specifically bound by the above substance immobilizing method is described with reference to FIG. 10. In order to detect target DNA 9008, labeled DNA 9009 binding to ruthenium complex (Ru complex) 9010 is used as a labeled substance. For this labeled DNA 9009, a labeled DNA that specifically binds to target DNA 9008 is used.

After target DNA 9008 is bound to immobilized DNA 9007 by the above substance immobilizing method, labeled DNA 9009 binding to ruthenium complex 9010 is injected into container 9001. Subsequently, by performing steps similar to the steps of the above substance immobilizing method, labeled DNA 9009 and target DNA 9008 are specifically bound. Labeled DNA 9009 that finally does not specifically bind is removed by washing. As a result, when target DNA 9008 is present in the sample, a complex in which immobilized DNA 9007, target DNA 9008, labeled DNA 9009 and ruthenium complex 9010 bind is formed on immobilization region 9002 (see FIG. 10).

Subsequently, when photons are produced by electric field oxidizing ruthenium complex 9010, light having a wavelength of 620 nm is emitted from Ru for about 350 ns. By making Ru emit light repeatedly by electrochemical continuous emission and detecting the light by photomultiplier 9006, the detection of target DNA 9008 can be indirectly performed.

According to the substance immobilizing method of each embodiment described above, the binding reaction efficiency of a specifically binding substance can be improved. Therefore, according to such favorable embodiments of the present invention, the efficiency of the specific binding of a target substance to an immobilization region can be improved, so that a reaction process providing high reaction efficiency can be provided in immunoassay.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2007-208150, filed Aug. 9, 2007, which is hereby incorporated by reference herein in its entirety.

What is claimed is:

1. A substance immobilizing apparatus having a region on which an immobilized substance to which a target substance can specifically bind is immobilized,
comprising an external force applying unit for applying an external force for moving the target substance back and forth in a direction perpendicular to an in-plane direction of the region and contacting the target substance with the immobilized substance a plurality of times.

2. The substance immobilizing apparatus according to claim 1, comprising a removing unit for removing at least the target substance that is not specifically bound to the immobilized substance when the target substance is moved back and forth in the direction perpendicular to the in-plane direction of the region and contacted with the immobilized substance a plurality of times by the external force applying unit.

3. The substance immobilizing apparatus according to claim 1, comprising, as the external force applying unit, a first external force applying unit for applying an external force for drawing the target substance to the region and a second external force applying unit for applying an external force for drawing the target substance away from the region, wherein by alternately performing application of an external force by the first external force applying unit and application of an external force by the second external force applying unit, the target substance is moved back and forth in the direction perpendicular to the in-plane direction of the region and contacted with the immobilized substance a plurality of times.

4. The substance immobilizing apparatus according to claim 3, wherein the target substance comprises a magnetic material, and the external force applied by at least one of the first external force applying unit and the second external force applying unit is a magnetic force.

5. The substance immobilizing apparatus according to claim 3, wherein the target substance has a charge, and the external force applied by at least one of the first external force applying unit and the second external force applying unit is an electrostatic force.

6. The substance immobilizing apparatus according to claim 1, further comprising an external force applying unit for applying an external force for moving the target substance in the in-plane direction of the region.

7. The substance immobilizing apparatus according to claim 6, wherein the external force for moving the target substance in the in-plane direction of the region is a hydraulic pressure of a liquid.

8. The substance immobilizing apparatus according to claim 6, wherein the external force for moving the target substance in the in-plane direction of the region is a magnetic force.

9. The substance immobilizing apparatus according to claim 6, wherein the external force for moving the target substance in the in-plane direction of the region is an electrostatic force.

10. A substance detecting apparatus comprising the substance immobilizing apparatus according to claim 1 and a unit for detecting the target substance that specifically binds to the immobilized substance.

11. The substance detecting apparatus according to claim 10, wherein the target substance comprises a magnetic material, and the unit for detecting the target substance is a unit for detecting a magnetic field generated from the magnetic material.

12. The substance detecting apparatus according to claim 10, wherein the target substance comprises a phosphor, and the unit for detecting the target substance is a unit for detecting fluorescence generated from the phosphor.

13. The substance detecting apparatus according to claim 10, wherein the target substance comprises a radionuclide, and the unit for detecting the target substance is a unit for detecting radiation generated from the radionuclide.

14. The substance detecting apparatus according to claim 10, wherein the target substance comprises a ruthenium complex, and the unit for detecting the target substance is a unit for detecting light generated from the ruthenium complex.

15. The substance detecting apparatus according to claim 10, wherein the target substance comprises a light emitting substrate, and the unit for detecting the target substance is a unit for detecting light generated from the light emitting substrate.

16. The substance detecting apparatus according to claim 10, wherein by specifically binding the target substance to a biological substance, the biological substance is indirectly detected.

17. A substance immobilizing method comprising
a stage of preparing a container having a region on which an immobilized substance to which a target substance can specifically bind is immobilized, and a sample comprising the target substance;
a first step of injecting the sample into the container and drawing the target substance to a surface of the region;
a second step of moving the target substance that does not specifically bind to the immobilized substance when the target substance is drawn to the surface of the region in the first step, in a direction of drawing the target substance that does not specifically bind to the immobilized substance away from the surface of the region; and
a third step of specifically binding the target substance to the immobilized substance by drawing the target substance to the surface of the region again to contact the immobilized substance.

18. The substance immobilizing method according to claim 17, wherein the second step and the third step are alternately carried out a plurality of times to contact the target substance with the immobilized substance a plurality of times.

19. The substance immobilizing method according to claim 17, wherein in at least one step of the second step and the third step, the target substance is moved in an in-plane direction of the region.

20. The substance immobilizing method according to claim 17, further comprising a step of removing the target substance that does not specifically bind to the immobilized substance, and impurities in the sample from the region after the third step.
